(19) United States
(12) Patent Application Publication
(10) Pub. No.: US 2009/0228044 A1
(43) Pub. Date: Sep. 10, 2009

(54) SYSTEMS AND METHODS FOR MOBILE SPINAL FIXATION RODS

(76) Inventors: Dong M. Jeon, Draper, UT (US); Patrick D. Moore, West Jordan, UT (US); Sang K. Lee, Seoul (KR)

Correspondence Address: MORRISS OBRYANT COMPAGNI, P.C., 734 EAST 200 SOUTH, SALT LAKE CITY, UT, 84102, US

(21) Appl. No.: 12/396,692

(22) Filed: Mar. 3, 2009

Related U.S. Application Data
(60) Provisional application No. 61/033,288, filed on Mar. 3, 2008.

(51) Int. Cl. A61B 17/70

(52) U.S. Cl. 606/254; 606/278

(57) ABSTRACT

Apparatus, systems and methods for mobile spinal fixation rod systems. A central housing may be formed in part from the enlarged end of a connection rod and may house a flexible insert in a cavity thereof. The enlarged head of a flexion rod may be disposed in the flexible insert and retained in the cavity by a coupler completing the central housing. A rod system of the present invention is capable of deflection, distraction and compression to preserve motion while providing fixation to the attached vertebrae.

### CROSS-REFERENCE TO RELATED APPLICATION

This application claims the benefit of U.S. Provisional Application No. 61/033,228, filed Mar. 3, 2008, the disclosure of which is incorporated herein by reference in its entirety.

### TECHNICAL FIELD

The present invention relates to devices and implants used in osteosynthesis and other orthopedic surgical procedures such as devices for use in spinal surgery, and, in particular, to a mobile spinal fixation rod system for effecting and maintaining a correction of the relative positions of the vertebrae and/or of the static and dynamic forces exerted on the vertebrae.

### BACKGROUND

Known spinal devices for reducing scoliotic deformations typically consist of components for anchoring in the vertebrae, such as hooks or intrapedicular screws, and rods or frames fixed to the anchoring components to impose a relative position on the various vertebrae. Such rigid or semi-rigid osteosynthesis devices produce a rigidification of the spinal column in the corrected position and are always associated with a bone graft for spinal fusion. Consequently, the fitting of such an osteosynthesis device has the effect of permanently suppressing the natural physiological mobility of the vertebrae. Thus, though such known osteosynthesis may, to a large extent, solve some of the problems connected with scoliotic deformation, they necessarily result in a handicap for the patient from the rigidification. Such rigidification is all the more serious because it is permanent and generally affects patients who are young and still growing. Accordingly, such devices may constitute an obstacle to the subsequent growth of the spine.

Further, such known osteosynthesis devices pose numerous problems with regard to the positioning and reliability of the anchoring components, which are subjected to high stresses because of the subsequent rigidity, and with regard to the fixing of the rods, plates or frames to the anchoring components, which must be carried out at the same time as the reduction of the deformation. Various semi-rigid osteosynthesis devices have been proposed in an attempt to resolve these drawbacks by preserving a certain elasticity which assists the fusion of the subsequent bone graft and facilitates the positioning of the anchoring components or reduces the stresses transmitted to the anchoring components. For example, FR-A-2 689 750, the contents of which are incorporated herein by reference, proposes an osteosynthesis device in which the rods have a flexibility with a high elastic limit. The elasticity thus preserved in the area of the fusion may assist the healing of the bone graft. Nevertheless, problems connected with the rigidification of the column persist after fusion with such a device. Similarly, U.S. Pat. No. 4,836,196, the contents of which are incorporated by reference herein, describes a spacing device disposed between anchoring components and a rigidification structure, making it possible to reduce the stresses transmitted between the vertebral body and the structure. U.S. Pat. No. 4,573,454, the contents of which are incorporated by reference herein, describes a device with an extensible structure consisting of a frame in two parts, one of which telescopes into the other, for the purpose of assisting subsequent growth in spite of the rigidification of the spine. Nevertheless, this problem is only partially solved since the portions of the spine fixed respectively to each of the parts of the structure are themselves rigidified without growth being possible.

In addition, conditions such as adjacent disk disease are a common complication resulting from solid fusion techniques. Adjacent disk disease may result from the additional lever force applied to the adjacent disks as a result of the solid fusion of the disk space and the affected vertebra bodies. This condition often requires additional surgeries to correct, which in turn further exacerbates the degeneration of adjacent disk spaces.

Accordingly there exists a need for assemblies and devices that address these problems by providing flexible stabilization. A system or assembly that provides dynamic stabilization to an adjacent vertebral body thus greatly reducing the lever force created from a newly created fusion mass at the time of the original surgery would be an improvement in the art.

### SUMMARY

Apparatus, systems and methods in accordance with the present invention are related to mobile spinal fixation rod systems. In one illustrative embodiment of a system in accordance with the present invention, a central housing may be formed in part from the enlarged end of a connection rod and may house a flexible insert in a cavity thereof. The enlarged head of a flexion rod may be disposed in the flexible insert and retained in the cavity by a coupler, completing the central housing. A rod system of the present invention is capable of deflection, distraction and compression to preserve motion while providing fixation to the attached vertebrae.

### DESCRIPTION OF THE DRAWINGS

It will be appreciated by those of ordinary skill in the art that the elements depicted in the various drawings are not necessarily to scale, but are for illustrative purposes only. The nature of the present invention, as well as other embodiments of the present invention may be more clearly understood by reference to the following detailed description of the invention, to the appended claims, and to the several drawings attached hereto.

FIG. 1 shows a sectional side view of an illustrative embodiment of a single-level mobile spinal fixation rod system.

FIGS. 2A, 2B and 2C are sectional side views of the embodiment of FIG. 1, depicted in various states of mobility.

### DESCRIPTION

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

Mobile spinal fixation rod systems in accordance with the present invention may preserve, at least in part, the natural physiological mobility of the vertebrae while effecting and maintaining a correction of the relative positions of the vertebrae with, or without the use of a graft/cage/spacer, or as not an adjunct to fusion. Such systems may use anchoring components/pedicle screws fixed to the vertebrae as a holding means.

Systems in accordance with the present invention may provide a dynamic means for a semi-restrained holding of the vertebrae with respect to each other in the corrected position while providing varying degrees of vertebral body freedom of movement. Such systems may result in a rod assembly that reduces the stress transmitted to the anchoring components fixed to the vertebrae to the lowest possible amount of mechanical stresses, to a magnitude which is strictly limited to that necessary for maintaining the correction of the deformation and/or applying the desired forces to the vertebrae.

Such systems may enable deformation of the spinal column to be reduced accurately in three dimensions and enable scoliosis to be reduced while preserving the natural physiological mobility, at least in part, of the vertebrae.

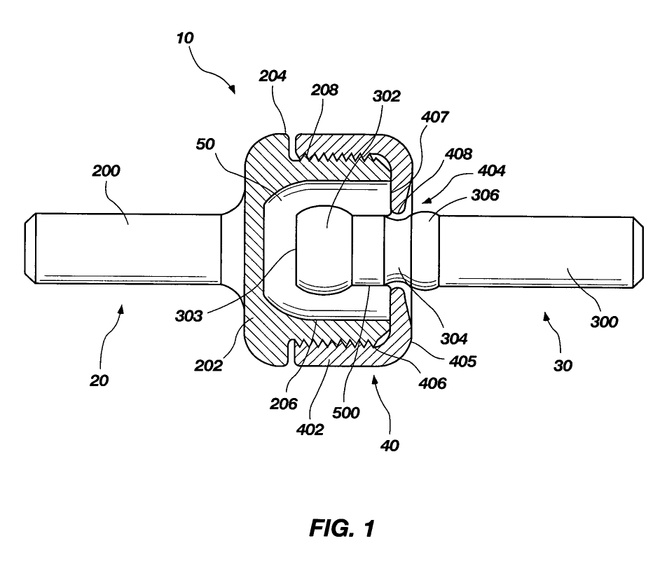

Referring generally to FIG. 1, there is shown one illustrative embodiment of a mobile spinal fixation rod system 10. System 10 includes two rod portions extending opposite one another from a central housing and may be formed from the following illustrative components: a connection rod 20, a flexion rod 30, a housing coupler 40, and a flexible insert 50.

Connection rod 20 includes a rod portion 200 formed as a rod, which may have a cylindrical cross-section extending from an enlarged proximal connector end 202 to a distal end. Proximal connector end 202 may be formed as a body having a flat front end, with a flexion cavity 206 opening therefrom into the body in the space formed by the encircling sidewall. Flexion cavity 206 may have a flat rear surface. The outer surface of the sidewall may be threaded 208 to accept a counterpart threading on a housing coupler 40. The threaded portion 208 of the sidewall may be recessed, as depicted by shoulder or step 204 to result in a housing structure having a fairly uniform cross-sectional thickness.

A flexible insert 50 may be disposed in flexion cavity 206. Flexible insert 50 may be formed from any suitable biocompatible material having appropriate elastic and flexion qualities. Some examples of suitable materials may include silicone based flexible materials and polymeric materials. Flexible insert 50 is shaped and sized to fit snuggly within the flexion cavity 206. An insertion space 500 may be formed in the flexible insert 50 for insertion of the flexion rod 30 therein. The insertion space 500 may be shaped to correspond to the proximal end of the flexion rod 30. For example, where the flexion rod 30 has a proximal end with an enlarged head, the flexible insert 50 may include an insertion space with an interior cavity sized formed about the same size as, or slightly smaller than the enlarged head, which is connected to the front of the flexible insert 50 by a narrower channel or “throat”, which has a diameter about the same size as, or slightly smaller than the rod portion of the flexion rod 30, to thereby assist in retaining the rod therein.

Flexion rod 30 is formed as a rod 300, which may have a cylindrical cross-section, which extends from a proximal end for placement in flexion cavity 206 to a distal end. The proximal end may have a flat end 303 which terminates the otherwise generally-spherical head 302. The flat front end 303 shortens the overall length of the spherical head 302 to result in a shorter connection, and may act to prevent puncture and premature wear of the flexible insert 50, compared to a spherical head having a spherical front end. A groove 304 may be disposed around the shaft of rod 30 distal to the spherical head 302. Groove 304 may have a curved concave cross-sectional shape. An enlarged section 306 of rod 30 may be formed distal to groove 30. The enlarged section 306 may appear as an encircling convex section or bump at the enlarged section 306, has a diameter greater than that of rod portion 300.

Housing coupler 40 may be formed as an open cylindrical body having a sidewall 402, the inner surface of which may include threads 406 for threaded attachment to the proximal connector end 202 of the connection rod 20. The front portion of housing coupler 40 has a planar rear surface 407 and a front surface 405. An opening 404 for passage of the flexion rod 30 shaft passes from the front surface 405 to the rear surface 407. The front surface 405 may be sloped towards opening 404 and the edges of the opening 404 may be formed as a curved lip 408. Upon installation, the rear surface 407 abuts the flexible insert 50, retaining it within the flexion cavity 206 and lip 408 may align with the groove 304 in the flexion rod 30.

It will be appreciated that although the shafts of both connection rod 20 and flexion rod 30 are depicted as having circular cross-sections, dependent on the usage, the cross-section may have a different cross-sectional shape, such as square or rectangular.

Connection rod 20, flexion rod 30, and housing coupler 40 may be constructed of any sturdy biocompatible material suitable for an orthopedic application. Suitable materials may include titanium, stainless steel, and alloys containing the same. Where these components are constructed from metallic materials, the materials may be similar, or identical to, the metallic materials used for the elongate fixation elements to avoid galvanic (mixed-metal) corrosion.

The shafts of connection rod 20 and flexion rod 30 may be constructed in various lengths based upon the intended usage of the particular system 10. For example, connection rod 20 will often be used to achieve spinal fusion and the length thereof will vary depending on the number of intervertebral spaces to be spanned for the fusion. By contrast, where a flexion rod 30 is intended to attach to only a single pedicle screw on a single vertebra, it may have length which is only slightly longer than the attachment mechanism or portion of the particular pedicle screw. As one illustrative example, where a pedicle screw having a round head with a diameter of about 14 mm that is configured for attachment to a rod is used, the shaft of the flexion rod 30 may have a length of from about 15 to about 17 mm, although this may vary dependent on the attachment device selected and the particular use.

In the depicted embodiment, the central housing formed by the enlarged connection end of the connection rod 20 and the housing coupler 40 may have a length of about 11 mm and a diameter of about 18 mm. The shafts of the connection rod 20 and flexion rod 30 may have diameters in the range of from about 5.0 to about 6.5 mm. It will be appreciated that these dimensions are only illustrative and embodiments may be built with these or other dimensions suitable for the particular use and the particular attachment devices.

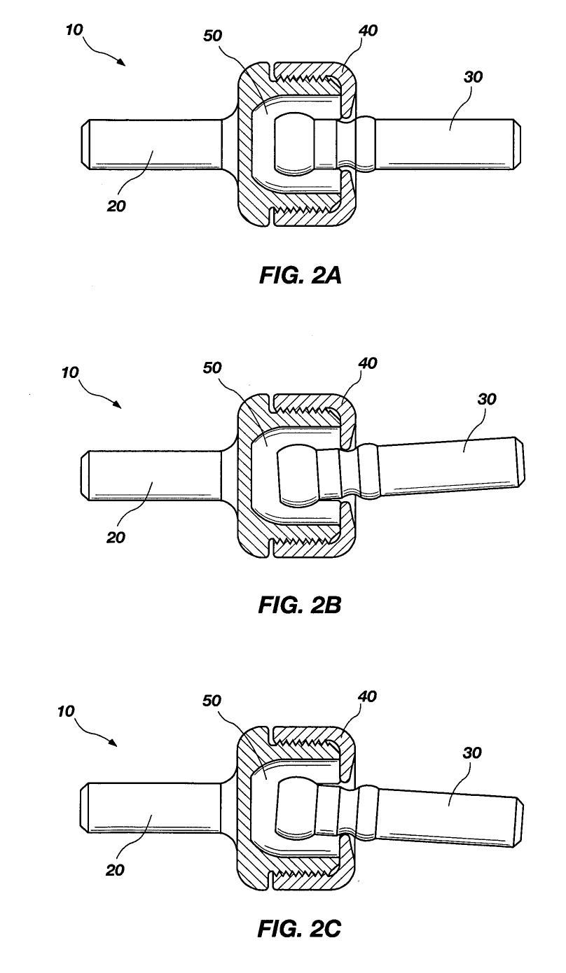

FIGS. 2A through 2C depict some the flexion ability of systems in accordance with the present invention. As depicted in FIG. 2A, upon installation, the axes of the shafts of the connection rod 20 and flexion rod 30 may align in a neutral position, with the long axes of the shafts generally parallel to one another. In some embodiments, the axes may be directly inline with one another. The flexible insert 50 allows flexion rod 30 to move therein, such that the shaft of the flexion rod 30 may deflect in any direction from alignment with the shaft of the connection rod 20. FIG. 2B depicts an upward deflection and FIG. 2C depicts a downward deflection, but it will be appreciated that deflection may occur in any direction around the 360 degree axis. The allowed angle of deflection may be up to about 6 degrees from the neutral position, although it is presently preferred to occur up to about 4 degrees.

Additionally, flexible insert 50 allows flexion rod 30 to be compressed into, or distracted from, the central housing to a desired distance. In the illustrated embodiment, it is presently preferred that the compression and distraction both be allowed to occur to about 2.0 mm from the neutral midpoint, providing an overall range of about 4.0 mm.

The alignment of convex groove 304 with the curved lip 408 of opening 404 accommodates the deflection of flexion rod 30. Additionally, the enlarged section 306 of the shaft of the flexion rod 30 prevents the rod 30 from slidably moving through the channel of an attachment structure, such as a pedicle screw. Either or both of these features may act to retain attachment and limiting deflection of the system to within desired parameters. Additionally, the spherical head 302 of flexion rod 30 is typically larger than opening 404, preventing withdrawal when the flexion rod 30 is distracted.

It will be appreciated that any systems in accordance with the present invention may be attached using any suitable pedicle screw or other bone attachment system, such as laminar hooks, known to those of ordinary skill in the art. The pedicle screws disclosed in U.S. published patent application no. 2007/0123870 (entitled BI-POLAR BONE SCREW ASSEMBLY) or U.S. published patent application no. 2007/0270839 (entitled MULTI-AXIAL DOUBLE LOCKING BONE SCREW ASSEMBLY), the contents of each of which are incorporated by reference herein, are presently preferred for such attachment.

Systems in accordance with the present invention may be oriented/placed during installation to hold the vertebrae in a corrected position against natural deforming forces and reduce the forces exerted on the vertebrae while preserving relative amounts of mobility, or as dynamic stabilization as an adjunct to fusion for treating a congenital or acquired deformation of the spine, in particular an idiopathic condition such as kypho-scoliosis, or a post-traumatic, tumorous, infectious, degenerative or other instability of the spine. Where appropriate, such systems can subsequently be removed, in particular at the end of the period of growth when the risks of a worsening or recurrence of the deformation or instability have disappeared.

The present invention also includes methods and procedures for effecting and maintaining, without osteosynthesis or graft for fusion, a correction of the relative positions of the vertebrae and/or of the forces exerted on the vertebrae for treating a congenital or acquired deformation of the spine, in particular an idiopathic deformation such as kypho-scoliosis, or a post-traumatic, tumorous, infectious, degenerative or other, instability of the spine, preserving at least in part the natural physiological mobility of the vertebrae, through use of systems in accordance with the present invention.

While the present invention has been shown and described in terms of preferred embodiments thereof, it will be understood that this invention is not limited to any particular embodiment and that changes and modifications may be made without departing from the true spirit and scope of the invention as defined and desired to be protected.

What is claimed is:

1. A mobile spinal fixation rod system comprising:
a connection rod having distal rod section and a larger proximal end, the larger proximal end comprising a sidewall which defines a cavity opening from a first surface at the proximal end;
a flexion rod having a distal rod section and a proximal connection end; the flexion rod including a groove disposed around a shaft of the rod distal to proximal;
a flexible insert formed from a flexible biocompatible material shaped and sized to fit snuggly within the cavity, the flexible insert including an insertion space formed within for receiving the proximal end of the flexion rod; and
a housing coupler formed as a cylindrical body having a front portion connecting a encircling sidewall defining a rear opening; the front portion having a opening therethrough to allow insertion of the distal rod section of the flexion rod therethrough and the encircling sidewall sized and configured to attach to an outer surface of the sidewall of the connection rod proximal end to retain the flexible insert and proximal connection end of the flexion rod in the connection rod cavity.

2. The mobile spinal fixation rod system of claim 1, wherein the connection rod proximal end includes a threading on at a least an outer portion of the sidewall to accept a counterpart threading on the inner surface of the encircling sidewall of the housing coupler.

3. The mobile spinal fixation rod system of claim 1, wherein the connection rod proximal end comprises a relatively larger distal section with a recessed proximal section having a relatively narrower diameter, such that when the housing coupler is disposed on the recessed proximal section the joined construct has a relatively uniform diameter across the entire connection rod proximal end.

4. The mobile spinal fixation rod system of claim 1, wherein the cavity in the connection rod proximal end has a flat rear surface.

5. The mobile spinal fixation rod system of claim 1, wherein the flexion rod proximal connection end comprises a head with a generally spherical portion terminating in a flat proximal end, the generally spherical portion of the head having a diameter greater that the size of the opening in the front surface of the housing coupler.

6. The mobile spinal fixation rod system of claim 1, wherein the flexion rod further comprises an enlarged section formed as an encircling bump distal to the groove.

7. The mobile spinal fixation rod system of claim 1, wherein the flexible insert includes the insertion space in the flexible insert is shaped to correspond to the proximal end of the flexion rod.

8. The mobile spinal fixation rod system of claim 1, wherein the front portion of the housing coupler defines a rear surface and a front surface, and the opening through the front portion is defined by a curved lip formed between the rear surface and front surface which aligns with the groove in the flexion rod upon installation.

9. The mobile spinal fixation rod system of claim 8, wherein a front surface of the housing coupler is sloped inward towards the opening through the front portion.

10. The mobile spinal fixation rod system of claim 1, wherein upon assembly, a long axis of the connection rod aligns with a long axis of the flexion rod in a neutral position.

11. The mobile spinal fixation rod system of claim 10, wherein upon assembly, the shaft of the flexion rod is capable of deflection from the neutral position in any direction around a 360 degree axis in an angle of up to about 4 degrees from the neutral position.

12. A mobile spinal fixation construct comprising:
a connection rod having distal rod section and a larger proximal end, the larger proximal end comprising a sidewall defining a cavity opening from a first surface at the proximal end;
a flexible insert formed from a flexible biocompatible material disposed within the cavity, the flexible insert including an insertion space;
a flexion rod having a distal rod section and a proximal connection end disposed in the insertion space of the flexible insert; the flexion rod including a groove disposed around a shaft of the rod distal to proximal; and
a housing coupler comprising a cylindrical body having a front portion connecting a encircling sidewall which is disposed on the outer surface of the sidewall of the connection rod proximal end, the front portion having a opening through which at least a portion of the distal rod section of the flexion rod passes.

13. The mobile spinal fixation construct of claim 12, wherein the housing coupler further comprises a threading on the inner surface of the encircling sidewall and is disposed on the connection rod proximal end by threaded attachment to a counterpart threading on at a least an outer portion of the sidewall of the connection rod proximal end.

14. The mobile spinal fixation construct of claim 12, wherein the connection rod proximal end comprises a relatively larger distal section with a recessed proximal section having a relatively narrower diameter and the housing coupler is disposed on the recessed proximal section.

15. The mobile spinal fixation construct of claim 12, wherein the cavity in the connection rod proximal end has a flat rear surface.

16. The mobile spinal fixation construct of claim 12, wherein the flexion rod proximal connection end comprises a head with a generally spherical portion terminating in a flat proximal end, the generally spherical portion of the head having a diameter greater that the size of the opening in the front surface of the housing coupler.

17. The mobile spinal fixation construct of claim 12, wherein the flexion rod further comprises an enlarged section formed as an encircling bump distal to the groove.

18. The mobile spinal fixation construct of claim 12, wherein the front portion of the housing coupler defines a rear surface and a front surface, and the opening through the front portion is defined by a curved lip formed between the rear surface and front surface which aligns with the groove in the flexion rod.

19. The mobile spinal fixation construct of claim 18, wherein a front surface of the housing coupler is sloped inward towards the opening through the front portion.

20. The mobile spinal fixation construct of claim 12, wherein a long axis of the connection rod aligns with a long axis of the flexion rod in a neutral position.

21. The mobile spinal fixation construct of claim 20, wherein the shaft of the flexion rod is capable of deflection from the neutral position in any direction around a 360 degree axis in an angle of up to about 4 degrees from the neutral position.

22. The mobile spinal fixation construct of claim 20, wherein the flexion rod is capable of being compressed into, or distracted from, the central housing by about 2.0 mm from a neutral midpoint, for an overall range of about 4.0 mm.
